(19) United States
(12) Patent Application Publication
(10) Pub. No.: US 2016/0220165 A1
(43) Pub. Date: Aug. 4, 2016

(54) DETECTING OLFACTORY MALINGERING

(71) Applicants: Safa Taherkhani, Tehran (IR); Farzad Taherkhani, Tehran (IR)

(72) Inventors: Safa Taherkhani, Tehran (IR); Farzad Taherkhani, Tehran (IR)

(21) Appl. No.: 15/097,084

(22) Filed: Apr. 12, 2016

Related U.S. Application Data
(60) Provisional application No. 62/186,376, filed on Jun. 30, 2015.

(51) Int. Cl. A61B 5/00

(52) U.S. Cl. CPC A61B 5/4011

(57) ABSTRACT

A test subject's answers to a forced-choice odorant test are received. The subject's answers are scored, based at least in part on identifying each of the subject's answers as correct or incorrect. The score includes a number of correct answers score and an answer pattern score. The subject is classified according to an olfactory condition type, which is a member of an olfactory condition set. The classifying is based at least in part on a combination of the number of correct answers score and the answer pattern score.

### CROSS REFERENCE TO RELATED APPLICATION

This application claims the benefit of priority from pending U.S. Provisional Patent Application Ser. No. 62/186,376, filed on 30 Jun., 2015, and entitled “Olfactory Malingering Detection Test (OMDT),” which is incorporated by reference herein in its entirety.

### TECHNICAL FIELD

The present application generally relates to the assessment of olfactory function, and particularly to olfactory malingering detection, and more particularly to designing a test for olfactory malingering detection.

### BACKGROUND

Olfactory malingering can be described as the intentional production of false or grossly exaggerated symptoms of anosmia. Olfactory malingering can be motivated by perceived incentives, such as receiving insurance settlements, or avoiding punishment, work, military service, jury duty, etc. For various purposes, for example, litigation, there can be a need to differentiate anosmic malingerers from actually anosmic patients.

There are known conventional testing techniques, generically referred to as smell identification test(s) (SIT), that are intended to differentiate malingering cases from anosmic cases. The University of Pennsylvania smell identification test (UPSIT) is one known SIT. UPSIT is a forced-choice test that consists of presenting a tester a set, e.g., approximately 40 of different odor samples, e.g., scratch- and sniff labels. The subject is given, with each odor sample, a list of choices. If the subject is anosmic the responses will be random, i.e., each choice has the same probability of being picked. For example, if the number of choices is Q and the subject is anosmic, then, for each odor sample, all Q choices have the same probability of being picked. Therefore, regardless of the anosmic subject picking answers at random, the probability of picking all or a very small number of incorrect answers is small. UPSIT exploits this, as it uses the count of correct answers in the test subject's responses to discriminate between the subject being anosmic and being a malingerer.

A problem with UPSIT is that it assumes subjects are truthful. Some subjects, though, can have both a motivation to deceive the UPSIT and familiarity with statistics and probability concepts, or the UPSIT classification scheme. Such a subject may deceive the UPSIT by intentionally picking wrong answers and right answers such that the count of correct answers is within the range statistically likely to be correct in an anosmic subject's responses. The UPSIT would then classify that malingering subject as anosmic.

One known technique intended to detect whether a subject is a malingering anosmic or is actually anosmic includes exposing the subject to irritants or trigeminal odorants, and asking for the subject's response. The intent is to exploit the fact that anosmic subjects, even though lacking an actual sense of smell, can sometimes detect irritants or trigeminal odorants. Accordingly, a subject classified by UPSIT as anosmic, and acknowledging irritants or trigeminal odorants, may likely be anosmic. In contrast, malingering anosmic subjects, although sensing the effects of irritants or trigeminal odorants, may deny detecting anything, on the belief that an answer of “yes” will reveal that the subject is cheating. A problem with this technique is that anosmic subjects may also deny sensing irritants or trigeminal odorants, fearing that a “yes” answer will result in not being classified as anosmic.

Accordingly, there is a need for an apparatus and method that can provide differentiation between malingering subjects and anosmic subjects, with at least a reduced probability of falsely classifying anosmic subjects as malingering subjects. There is also a need for an apparatus and method that, with a usable accuracy, can detect one or more answering strategies employed by malingering subjects to cheat known SITs.

### SUMMARY

The following brief summary is not intended to include all features and aspects of the present application, nor does it imply that practices must include all features and aspects discussed in this summary.

Features in one disclosed method according to one aspect can provide detection of olfactory malingering. Example operations can include presenting a subject a forced-choice odor identification test, receiving the subject's answers, and scoring the subject's answers. In an aspect, the scoring can be based at least in part on identifying each of the subject's answers as correct or incorrect, according to a number of correct answers score and an answer pattern score. In an aspect, example operation can further include classifying the subject according to an olfactory condition type, the olfactory condition type being a member of an olfactory condition set, the olfactory condition set including a malingering type. In an aspect, operations in classifying the subject can be based at least in part on a combination of the number of correct answers score and the answer pattern score.

Features in another disclosed method according to one aspect can also provide detection of olfactory malingering. Example operations can include presenting a subject a forced-choice odor identification test, receiving the subject's answers, identifying each of the subject's answers as correct or incorrect and generating, based at least in part on identifying each of the subject's answers as correct or incorrect, a number of correct answers score and at least one from among a number of consecutive correct answers score and a position of the first correct answer score. In an aspect, example operations can also include classifying the subject according to olfactory condition type, the olfactory condition type being a member of an olfactory condition set, the olfactory condition set including a malingering type. In an aspect, the classifying can be based at least in part on a combination of the number of correct answers score and a comparison of the number of consecutive correct answers score to a number of consecutive correct answers criterion, or a comparison of the position of the first correct answer score to a position of the first correct answer score criterion, or both.

Features in one disclosed data processing system, directed to detecting olfactory malingering, can comprise: a processor, and a memory, coupled to the processor, storing machine-readable executable instructions. In an aspect, the machine-readable executable instructions can be configured to cause, when executed by the processor, the processor to receive a subject's answers to an odor identification test, and score the subject's answers, based at least in part on identifying each of the subject's answers as correct or incorrect. In an aspect, the machine-readable executable instructions can be configured to cause the processor, when executed, to score the subject's answers according to a number of correct answers score and an answer pattern score. In an aspect, the machine-readable executable instructions can be configured to cause the processor, when executed, to classify the subject according to an olfactory condition type, the olfactory condition type being a member of an olfactory condition set, based at least in part on a combination of the number of correct answers score and the answer pattern score, with the olfactory condition set including a malingering type.

### BRIEF DESCRIPTION OF THE DRAWINGS

While the specification concludes with claims particularly pointing out and distinctly claiming the subject matter that is regarded as forming the present application, it is believed that the application will be better understood from the following description taken in conjunction with the accompanying DRAWINGS, where like reference numerals designate like structural and other elements, in which:

FIG. 1 shows a logical flow of example operations in a process in one olfactory malingering detection test (OMDT) method according to various aspects.

FIG. 2A shows one exemplary test card for presentation to a subject, in one implementation of a process according to various aspects.

FIG. 2B one exemplary test booklet, in one implementation.

FIG. 3 shows one example relation of probability of the occurrence of different scores obtained base on the criterion of number of consecutive correct answers.

FIG. 4 shows one example relation of probability of the first correct answer occurring after answering a certain number of questions.

FIG. 5 shows one example of one form for a probability density function of the number of similar wrong answers chosen for a specific odorant.

FIG. 6 illustrates one example of one answer key of one forced-choice odor identification test according to various aspects.

FIG. 7 illustrates another exemplary answer sheet.

FIG. 8 illustrates one exemplary decision scheme for classifying a subject, based at least in part on scores of the subject's answers to one forced-choice odor identification test according to various aspects.

FIG. 9 illustrates another exemplary decision scheme for classifying a subject based, at least in part, on scores of the subject's client answers to one forced-choice odor identification test according to various aspects.

FIG. 10 is a block diagram of one data processing system.

### DETAILED DESCRIPTION

Aspects and features, and examples of various practices and applications are disclosed in the following description and related drawings. Alternatives to disclosed examples may be devised without departing from disclosed concepts.

The terminology used herein is for the purpose of describing particular examples and is not intended to impose any limit on the scope of the appended claims.

Certain examples are disclosed, explicitly or implicitly, as using components or operations taken or adapted from known, conventional techniques. Such components and operations will not be described in detail or will be omitted, except where incidental to example features and operations, to avoid obscuring relevant details.

The words “exemplar” and “exemplary,” as used herein, are interchangeable and mean “serving as an example, instance, or illustration.” Any feature or aspect described herein as “exemplar” or “exemplary” is not necessarily preferred or advantageous over other features or aspects. Description of a feature, advantage or mode of operation in relation to an aspect, or to an example combination of aspects, is not intended to convey that all practices that include the aspect or the combination also include the discussed feature, advantage or mode of operation.

As used herein, the singular forms “a”, “an” and “the” are intended to include the plural forms as well, unless the context clearly indicates otherwise. The terms “comprises”, “comprising,”, “includes” and/or “including”, as used herein, specify the presence of stated features, integers, steps, operations, elements, and/or components, but do not preclude the presence or addition of one or more other features, integers, steps, operations, elements, components, and/or groups thereof.

Labels used herein such as, without limitation, “first” and “second” may be used solely to distinguish one structure, component, operand, action or operation from another without necessarily requiring or implying any order in time or in importance.

Unless explicitly stated, or the context clearly indicates otherwise, description of an example implementation of a feature, together with description of an example alternative implementation, does not mean that the example and alternative example cannot be used in combination.

The term “forced-choice,” as used herein, means a test that requires the test-taker to identify or indicate identification of a previously-presented stimulus, e.g., an odorant, by choosing between a finite number of alternative choices.

The term “question,” as used herein, except where otherwise stated or where made clear form its context to mean otherwise, means a presentation to a subject of an odorant sample, along with a means for the subject to choose, in response, between a finite number of alternative choices.

The term “normosmic subject,” as used herein, means a subject with a normal sense of smell, where “normal” means within a range of acuity that would be understood as “normal” by a person of ordinary skill in the art.

The term “microsmic subject,” as used herein, means a subject with diminished, i.e., less than normal sense of smell, either to all odorants or to specific odorants.

The “anosmic” subject, as used herein, means a subject with diminished, i.e., less than normal sense of smell, either to all odorants or to specific odorants

The term “odorant,” as used herein, means a substance that has or emits a smell that is likely detectable by and describable by a normosmic subject of a culture with which the subject is familiar, when the normosmic subject is exposed to the odorant through exposure techniques described or referenced herein. The term “odorant,” as used herein, encompasses substances that are natural or synthetic, or both. As one non-limiting illustration, an odorant that has or emits a smell that a normosmic subject, having prior knowledge of the smell of oranges, would identify as the smell of an orange, can comprise a natural extract of oranges, or a synthetic substance, or both.

Methods disclosed herein can be directed to detecting and discriminating at least between anosmic subjects and olfactory malingering subjects.

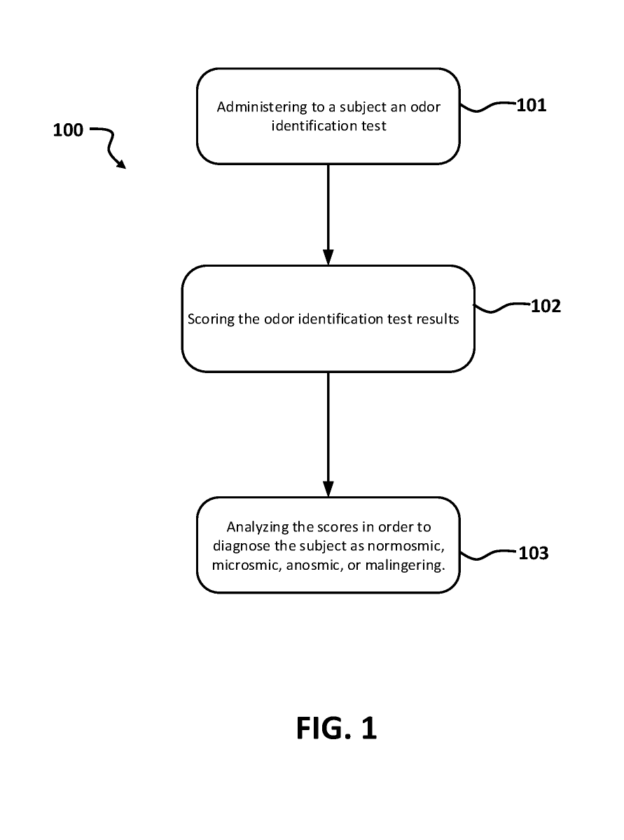

FIG. 1 is a block diagram representing one example flow 100 of operations in one process in one method according to various aspects. One example execution of the flow 100 can begin at 101 where operations can include administering to a subject a forced-choice odor identification test. In an aspect, operations at 101 can include presenting the subject with a sequence of odorants and, associated with each presentation, presenting the subject a list of alternative choices, and then receiving the subject's selection.

In an aspect, operations at 101 can be performed, at least in part, on or through a subject test interface apparatus (not explicitly visible on FIG. 1). One subject test interface apparatus can comprise fixing the odorants on scratch-and-sniff labels. In an aspect, the scratch-and-sniff labels can be implemented on cards. The cards, which can be referred to as “test cards,” can include a printed medium showing list of alternative choices, and adjacent each choice in the list a manually writable field, for example, a check box. The subject test interface apparatus can further include a check box scanner, for example, a commercially available multiple-choice test scanner, or an adaptation of same, connected to or accessible by a general purpose programmable computer. In an aspect, the general purpose programmable computer can include a processor engine coupled to a memory resource. The test results, for example, from the scanner, can be stored in the memory resource. In a further aspect, machine-readable instructions can also be stored in the memory resource that, when executed by the processor engine, cause the processor engine to perform remaining operations in the flow 100, such as described later in greater detail.

It will be understood that “subject test interface apparatus” can be a logical feature distributed over a plurality of devices. For example, the odor samples may be provided by scratch and sniff cards, as described above, and the subject's responses can be received, for example, on a touch-screen coupled to the general purpose programmable computer. FIG. 1).

Regarding the scope of odorants, methods and apparatuses according to the disclosed concepts have no limitation on the scope of odorants that can be used.

For example, without limitation, odorants used in practices according to the disclosed concepts can include any or more for, for example, banana, rose water, cinnamon, gasoline, apple, saffron, mint, coffee, cologne, cantaloupe, garlic, cucumber, swage, smoke, sausage, vinegar, oil, orange, onion, bread, jasmine, strawberries, chocolate, fish, cigarette, natural gas, alcohol, lemon, pizza, peanuts, lilac, bubble gum, watermelon, tomato, menthol, honey, lime, cherry, grass, motor oil, pineapple, cola, chili, leather, coconut, cedar, soap, pumpkin pie, cheddar cheese, paint thinner, pine, rose, peach, black pepper, gingerbread, turpentine, musk, and grass. The list of odors can involve essentially any odor.

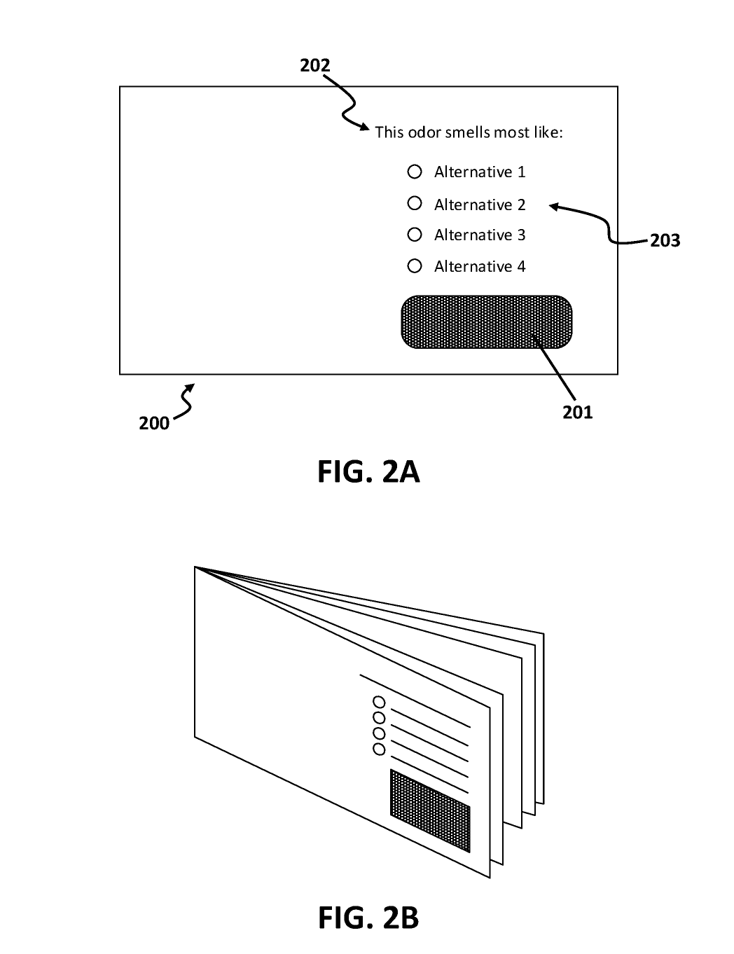

FIG. 2A shows a projection of one example test card 200, as may be seen from a perspective of a subject holding it. Referring to FIG. 2A, the test card 200 can include a scratch-and-sniff label 201, as described above. The test card 200 can also include, for example on a printed medium, a question stem 202 showing a plurality of alternative choices 203. The plurality of alternative choices 203 can include at least 2 alternative choices. One of the alternatives choices 203 is the correct answer, and other alternatives choices are incorrect. In an aspect, the incorrect alternative choices can function as distracters, as will be described in greater detail later. Adjacent each of the alternative choices 203 may be a check box (visible but not separately labeled) on, for example, with a writable medium. In an aspect, the test card 200 can be configured as compatible with a multiple-choice test sheet scanning apparatus, as described above.

FIG. 2B shows a booklet comprising set of test cards 200 each, for example, being an individual page of the booklet. An example test booklet is illustrated in FIG. 2B.

It will be understood that the test card 200 is only one example implementation for presenting odorant samples to the subject, and receiving the subject's responses. In one alternative, the odorants can be presented by smell bottles (not explicitly visible in the figures). Persons of ordinary skill, upon reading the present disclosure, may identify various other techniques or devices for presenting odorant to the subject, or for receiving the subject's responses, or both.

Referring to FIG. 1, in an aspect, operations at 101 can include presenting the subject with only a sub-set or sub-plurality of odorants from a larger universe of odorants. The sub-set or sub-plurality can be referenced, for purposes of description, as a “sample set of odorants.” In an aspect, operations at 101 can present the sample set of odorants to the subject according to a repetition pattern. The repetition pattern can include, for example, presenting the subject 2 instances of each odorant in the sample set of odorants. In an aspect, the repetition pattern can include a wrong answer pattern. In a related aspect, the wrong answer pattern can be configured such that, for each odorant test-response question, the list of alternative choices includes specific wrong alternative choices. For purposes of description, the specific wrong alternative choices will be alternatively referred to as “distracters.”

Referring to FIG. 1, after operations at 101 the flow 100 can proceed to 102, where operations can compare the subject's responses to the forced-choice odor identification test to a set of criteria can be performed and, based at least in part on passing or failing the criteria, a score can be generated. The criteria at 102 can include a number of correct answers criterion, and at least one answer pattern criterion. In an aspect, the at least one answer pattern criterion, or answer pattern criteria, can comprise at least one from among distribution of correct answers criterion, number of consecutive correct answers criterion, position of the first correct answer criterion, distribution of correct answers for a specific odorant criterion, and number of similar wrong answers chosen for a specific odorant criterion.

Operations at 102 can generate the score as an indication of whether the subject's responses pass the number of correct answers criterion, in combination with an answer pattern criteria pass/fail count. As will be described in greater detail later, subsequent operations, for example, at 103, can then discriminate the subject, based at least in part on the score generated at 102, between types that characterize acuity (or lack of same) of sense of smell. For purposes of description, the type will be referred to as “olfactory types.” In an aspect, there can be a set of olfactory types. The set can include at least anosmia and malingering subject. The terms “malingering subject” and “malingering,” as used herein, means a subject that actually has zero or an insignificant loss of sense of smell, but tries to convince others that he or she is anosmic.

Exemplary features and relating to the number of correct answers criterion will now be described. In an aspect, the number of correct answers criterion can be based, at least in part, on a correct answer reference range. The correct answer reference range can be a range of correct answer numbers that would be statistically likely in an anosmic subject response to a given forced-choice odor identification test. If the correct answer count is within the anosmic correct answer reference range, the count passes the anosmic correct answer criterion. If it does not, the count fails the anosmic correct answer criterion.

Regarding the numerical values of “statistically likely,” in the context of the range of correct answer count in an anosmic subject's responses, persons of ordinary skill having possession of this disclosure and facing a given application can readily determine such numerical values, without undue experimentation, for practicing according to disclosed concepts. Further detailed description of determining such values is therefore omitted.

For illustration, an example of an anosmic correct answer reference range, and anosmic correct answer criterion, will assume a forced-choice odor identification test having 40 questions, and 4 alternative choices for each. It may be empirically determined (for a given target error rate and a given target FPR) that the anosmic correct answer reference range spans, for example, from 8 to 12. Accordingly, if a subject's responses to the above-described example forced-choice odor identification test have a correct answer count in the range of 8 to 12, the responses pass the anosmic correct answer criterion. If not, the subject's responses fail the anosmic correct answer criterion.

Specific example calculations and results for one example anosmic correct answer reference range, and corresponding anosmic correct answer criterion, are described later in this disclosure, under the header “Number of Correct Answers,” and in reference to Table 1.

Features and aspects of example answer pattern criteria, and in comparing the results obtained at 101 to example answer pattern criteria, will now be described.

In an aspect, one of the answer pattern criteria can be referred to as a “distribution of correct answers criterion.” The distribution of correct answers criterion can be based on consistency in the number of correct answers provided by a subject in response to groups of questions. Example operations in determining, and comparing the results obtained at 101 to the distribution of correct answers criterion are described in greater detail under the header “Distribution of Correct Answer,” and elsewhere in this disclosure.

In an aspect, another of the answer pattern criteria, against which the results obtained at 101 be compared, which for description purposes can be referred to as a “position of the first correct answer distribution criterion,” will now be described. In overview, the position of the first correct answer distribution criterion can exploit an observation by the present inventors that malingering subjects tend to avoid answering early questions correctly, and then try to place their first correct answer after providing incorrect answers to a couple of the questions. The present inventors believe, without subscribing to any particular scientific theory, that malinger subjects' motivation may be fear of detection if their first correct answer occurs in an early iteration. Example operations in determining, and in utilizing the position of the first correct answer distribution criterion are described in greater detail under the header “Position of the First Correct” and elsewhere later in this disclosure.

Each of the answer pattern criteria described are determined, and the results of operations at 101 are compared to same, without consideration of detecting answer patterns that correlate with specific odorants. Such answer pattern criteria can be referenced, for description purposes, as “general answer pattern criteria.” In an aspect, the answer pattern criteria can comprise, either in combination with or not in combination with any of the above-described general answer pattern criteria, at least one answer pattern criterion that relates to one or more specific odorants presented to the subject. Such an answer pattern criterion, or criteria can be referenced, for description purposes of description, as an “odorant-specific answer pattern criterion” or “odorant-specific answer pattern criteria.”

One odorant-specific answer pattern criterion against which the results obtained at 101 can be compared, which will be referred to as “distribution of correct answers chosen for a specific odorant criterion,” will now be described. In an aspect, the distribution of correct answers chosen for a specific odorant criterion can be determined and utilized similarly to the distribution of correct answers described above, except that it focuses on responses to a specific odorant. Example operations in determining, and utilizing the distribution of correct answers chosen for a specific odorant criterion are described in greater detail, under the header “Distribution of Correct Answers Chosen for a Specific Odorant,” and elsewhere later in this disclosure.

Another odorant-specific answer pattern criterion against which the results obtained at 101 be compared, which will be referred to as “number of similar wrong answers chosen for a specific odorant criterion,” will now be described. In an aspect, the odorant test questions can be arranged such that each odorant is presented to the test subject in a repeated manner. The repeated manner can be configured, for example, such that each odorant is presented to the test subject at least 2 times. The operations of presenting the questions can be configured, according to an aspect, such that repeated instances of the same odorant use the same list of alternative choices. For example, if test cards, such as described in reference to FIG. 2B, are employed, each test card having the same odorant sample microencapsulated in its scratch-and-sniff label 201 will also have the same list of alternative choices at 203. Benefits of this feature can include utilization of a tendency, identified by the present inventors, that the number of similar wrong answers chosen for a specific odorant criterion can correlate with whether the subject is anosmic or malingering. Such tendency identified includes, without subscribing to any particular scientific theory, a tendency of malingering subjects to choose a specific wrong answer whenever the subject smells a specific odorant. In addition, without subscribing to any particular scientific theory, such tendency identified includes anosmic subjects being unlikely to pick the same specific wrong alternative or distracter in response to repeated exposures to a specific odorant.

Example operations in determining, and in utilizing the number of similar wrong answers chosen for a specific odorant criterion are described in greater detail, under the header “Number of Similar Wrong Answers Chosen for a Specific Odorant,” and elsewhere later in this disclosure.

In an aspect, operations at 102 can be configured to provide what will be termed, for purposes of description, a “response criteria score, the above-described score can be configured such that the results of operations 101 are compared to the

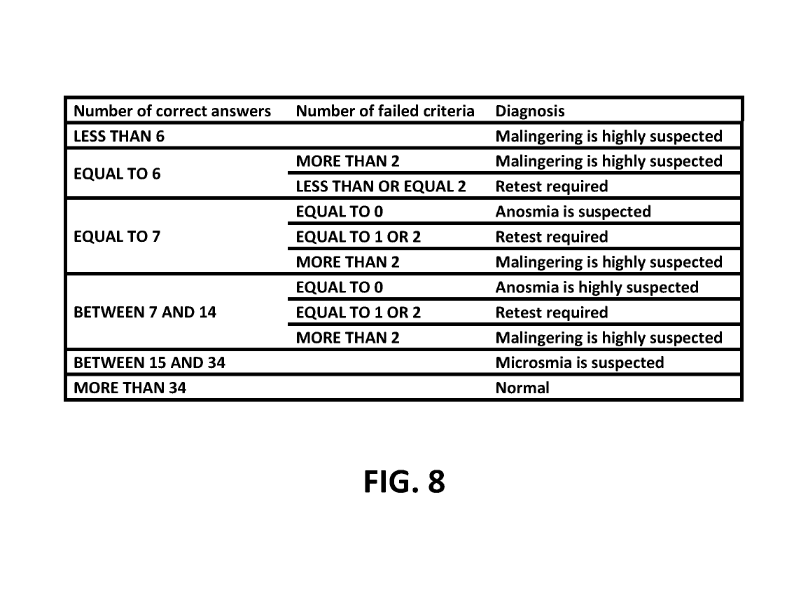

Referring to FIG. 1, after completing operations at 102, the flow 100 can proceed to 103 where operations can be applied to the results of operations at 102 and can generate, in response, a classification of the test subject into classes that can include at least anosmic and malingering. In an aspect Examples of such operations at 103 are described in greater detail in reference to FIG. 8 and elsewhere later in this disclosure. As will also be described in greater detail, operations at 103 can be further configured to classification of the test subject into classes that can include at least anosmic and malingering, as normosmic, microsmic, anosmic, or malingering.

### Number of Correct Answers

As described in reference to FIG. 1, the number of correct answers score can be based on the number of correct answers, i.e., number of times the subject identifies an odorant correctly. For a forced-choice odor identification test with n questions, the probability of getting k correct answers by randomly answering the questions can be calculated by a probability mass function, such as the following Equation (1):

$$p ( k ) = ( \begin{matrix}n \\ k\end{matrix} ) {{p}^{k} ( 1 - p )}^{n - k} \qquad Equation ( 1 )$$

where p(k) is the probability of randomly getting k correct answers in a forced-choice odor identification test comprising n questions, and p is the probability of choosing the correct answer to a single question. The value of p can be determined by the number of alternative choices. For example, for a question with four alternatives, p is equal 0.25.

A chance level can be calculated for the number of correct answers in the forced-choice odor identification test, based on the probability of getting k correct answers by randomly answering the questions, which is discussed hereinabove. For example, for a forced-choice odor identification test with 40 questions, and four alternatives given for each question, the probability of getting k correct answers, assuming the answers to be random, can be calculated by plugging n equal to 40 and p equal to 0.25 into Equation (1). The associated arithmetic can be represented by the following Equation (2), which is Equation (1) with the example values above plugged in:

$$p ( k ) = ( \begin{matrix}40 \\ k\end{matrix} ) {( 0.25 )}^{k} {( 1 - 0.25 )}^{40 - k} \qquad Equation ( 2 )$$

Table 1 presents with 15 rows, the top row corresponding to k=zero, followed by 14 rows fork ranging from one to 14, proceeding downward with increasing values of k, the values of p(k) generated by Equation (2). The values of k are in the first (leftmost) column, and the values of p(k) are in the fifth (rightmost) column. Equation (2), as described above, assumes for a forced-choice odor identification test with 40 questions, and 4 alternatives choices given for each question. Therefore, the values of n equal 40, p equal 0.25, and 1-p equal to 0.75, are constants, shown in the second, third, and fourth columns, respectively.

TABLE 1

| k | n | p | 1 − p | p(k) |
|---|---|---|---|---|
| 0 | 40 | 0.25 | 0.75 | 0.0000100566 |
| 1 | 40 | 0.25 | 0.75 | 0.0001340878 |
| 2 | 40 | 0.25 | 0.75 | 0.0008715707 |
| 3 | 40 | 0.25 | 0.75 | 0.0036799652 |
| 4 | 40 | 0.25 | 0.75 | 0.0113465595 |
| 5 | 40 | 0.25 | 0.75 | 0.0272317428 |
| 6 | 40 | 0.25 | 0.75 | 0.0529506109 |
| 7 | 40 | 0.25 | 0.75 | 0.0857295605 |
| 8 | 40 | 0.25 | 0.75 | 0.1178781457 |
| 9 | 40 | 0.25 | 0.75 | 0.1397074320 |
| 10 | 40 | 0.25 | 0.75 | 0.1443643464 |
| 11 | 40 | 0.25 | 0.75 | 0.1312403149 |
| 12 | 40 | 0.25 | 0.75 | 0.1057213648 |
| 13 | 40 | 0.25 | 0.75 | 0.0759025183 |
| 14 | 40 | 0.25 | 0.75 | 0.0487944760 |

| | |
|---|---|
| Σᵢ₌₆¹⁴pᵢ (k) | 0.9022887694 |

The bottom row of Table 1 shows a summation of p(k), for k ranging from six to 14, being equal to 0.9022887694. The means that the probability of the correct answer count, in responses by an anosmic subject taking the example forced-choice odor identification test, being in the range of six to 14 is 0.9022887694. For this example, the probability value 0.9022887694 will be assumed to “statistically likely.” Accordingly, for this example, the “number of correct answers criterion” can be defined as follows: the results from the subject taking the example forced-choice odor identification test having a number of correct answers count in the range of 6 to 14. Stated differently, with k being the number of correct answers, the “number of correct answers criterion” can be k being in the range of 6 to 14.

As described in the Background of this disclosure, using the number of correct answers criterion alone to discriminate anosmic subjects from normosmic subjects is a known conventional technique, of which UPSIT is an example. However, as also described in the Background, a problem with these techniques, including UPSIT, is that they assume subjects are truthful. Some subjects, though, having certain motivations and knowledge may attempt to deceive the test (including UPSIT) by intentionally picking wrong answers and right answers. For example, assuming the UPSIT classification scheme described above, a malingering subject may choose his answers such that 8 to 12 are correct. UPSIT and other techniques using only the number of correct answers criterion would then classify that malingering subject as anosmic.

### Distribution of Correct Answers

As described above, one of the answer pattern criteria can be the distribution of correct answers criterion. In an aspect, the distribution of correct answers criterion can be configured such that a subject's responses to groups of questions will pass if the show a low variation in the number of correct answers from group to group.

In an aspect, distribution of correct answers can be determined by presenting the n questions to the subject as a plurality of groups, and then calculating a coefficient of variation, “Cv,” which is one metric of variation in the number of correct answers from group to group. The coefficient of variation can be defined as the ratio of the standard deviation of the number of correct answers in each group to the average number of correct answers in each group. The coefficient of variation can be calculated according to the following Equations (3) to (6)

$$\begin{matrix}Cv = \frac{σ}{\overline{x}} & Equation ( 3 ) \\ 0 \leq Cv \leq {T}^{1 / 2} & Equation ( 4 ) \\ \overline{x} = \frac{1}{T} {\sum }_{i = 1}^{T} {x}_{i} & Equation ( 5 ) \\ σ = {( \frac{1}{T} {\sum }_{i = 1}^{T} {( {x}_{i} - \overline{x} )}^{2} )}^{\frac{1}{2}} & Equation ( 6 )\end{matrix}$$

where Cv represents the coefficient of variation, T represents the number of groups (e.g., booklets), x̅ represents the average number of correct answers x̅, σ represents the standard deviation of the number of correct answers in each group (e.g., each booklet), and x represents the number of correct answers in each group.

Assuming an arrangement of the forced-choice odor identification test that presents the subject with integer T groups of questions, each group can have integer U questions (e.g., integer T booklets, each having integer U test cards). The sample space for all possible numbers of correct answers in the T groups can be calculated using Equation (7), as follows:

SSP={(x₁,x₂ . . . x_{T})|0≦xᵢ≦U,Σᵢ₌₁^{T}xᵢ≠0} Equation (7)

where x₁, x₂ . . . x_{T} denote the number of correct answers chosen by the subject in each of the T groups.

The probability of each set to occur can be calculated using the following Equations (8) and (9):

$$\begin{matrix}{set}_{jth} = \{ {( {x}_{1} , {x}_{2} … {x}_{T} )}_{i} \} i = 1 , 2 , 3 … N & Equation ( 8 ) \\ P ( {set}_{jth} ) = {\sum }_{i = 1}^{N} \{ {\prod }_{i = 1}^{T} ( \frac{U}{{x}_{k}^{i}} ) {( \frac{1}{Q} )}^{k} {( \frac{Q - 1}{Q} )}^{U - k} \} & Equation ( 9 )\end{matrix}$$

Table 2 shows presents a plurality of coefficient of variations for a selection of sample cases in a 40-question forced-choice odor identification test, each question having 4 alternative choices, with the questions divided into 4 groups, each group containing 10 of the questions. The 4 groups can each comprise a booklet, each booklet containing 10 test cards, configured described in reference to FIGS. 2A and 2B. The values were determined, in part by applying calculations according to Equations (3) through (9), with U equal 10, Q equal 4, and T equal 4. Each entry in the first (leftmost) column is the total number of correct answers provided by the subject in the entire test. In the second column a few exemplar sets are presented that show the number of correct answers in each booklet.

For example, referring to Table 2, entries {0, 0, 0, 8} in the second column means a case where no correct answers are given by the subject in the first 3 booklets, and 8 correct answers in the last booklet. The third column presents the coefficient of variation for each example case.

TABLE 2
Coefficient of Variation (Cv) for One Example

| No. of correct answers | Distribution of correct answers in booklets | Cv |
|---|---|---|
| 8 | {0, 0, 0, 8} | 2 |
| 8 | {0, 0, 8, 0} | 2 |
| 12 | {0, 0, 0, 12} | 2 |
| 12 | {3, 3, 3, 3} | 0 |
| 8 | {2, 2, 2, 2} | 0 |
| 10 | {5, 1, 1, 3} | 0.77 |
| 10 | {0, 7, 1, 2} | 1.24 |
| 10 | {1, 3, 4, 2} | 0.52 |

For purposes of illustration, it will be assumed for this example that a coefficient of variation of approximately 2 or greater indicates correct answers are not distributed evenly over the 4 groups. In other words, over the course of the entire test, the subject shows a statistically significant variation in the rate of correct answers. The present inventors have identified, without subscribing to any particular scientific theory, a correlation between uneven distribution of correct answers and the answers being obtained from malingering subjects. The present inventors have also identified, without subscribing to any particular scientific theory, a correlation between even distribution of correct answers and anosmia.

It will be assumed for this example that a coefficient of variation near zero indicates that the correct answers are distributed evenly over the 4 groups. A reference range for the coefficient of variation can be obtained by calculating the probability of each coefficient of variation in responses from known anosmic subjects. In other words, the responses are known as being random. For example, simulated random responses to a 40-item forced-choice odor identification test with 4-choice questions, where the questions are divided into 4 groups (e.g., 4 booklets), show highly probability of the coefficient of variation being between 0 and 0.86. Accordingly, for this example forced-choice odor identification test, a distribution of correct answers criterions can be set as follows: a coefficient of variation between 0 and 0.86.

### Number of Consecutive Correct Answers

As described above, another of the answer pattern criteria can be the number of consecutive correct answers criterion. The criterion can utilize the tendency, identified by the present inventors, that random answers to forced-choice odor identification test questions, such as the answers provided by anosmic subjects (since, by definition, they cannot identify the odorants correctly) are unlikely to have a plurality of consecutive correct answers. The present inventors have identified, without subscribing to any particular scientific theory, that answers provided by malingering subjects tend to show a higher incidence of consecutive correct answers. One score for the incidence of consecutive correct answers can be the “number of consecutive correct answers score” that can be defined, for example, according to the following Equation (10):

$${F}_{3} = \frac{Number of two consecutive correct answers}{Total number of correct answers} \\ 0 \leq {F}_{3} < 1 \qquad Equation ( 10 )$$

As can be seen, operations implementing to Equation (10) can be straightforward, namely, counting the instances of 2 consecutive correct answers, and then dividing the count by the total number of correct answers. Table 3 below shows example calculations of F₃.

TABLE 3
Example Scoring of the Number of Consecutive Correct Answers.

| No. of Correct | Questions to Which a | | |
|---|---|---|---|
| Answers | Correct Answer is Given | Calculation | F₃ |
| 8 | 11-12-18-28-29-30-38-39 | 4/8 | 0.5 |
| 8 | 11-13-18-28-30-35-37-39 | 0 | 0 |
| 7 | 3-11-12-20-21-32-40 | 2/7 | 0.286 |
| 6 | 11-12-32-33-34-35 | 4/6 | 0.667 |
| 6 | 10-11-23-24-34-35 | 3/6 | 0.5 |

Referring to Table 3, the first column shows the total number of questions answered correctly; and the second column identifies the question numbers to which the subject gave a correct answer. For example, “3-11-12-20-21-32-40” means that the subject has correctly answered questions 3, 11, 12, 20, 21, 32, and 40. The answers to questions 11 and 12 are consecutive and those to questions 20 and 21 are consecutive. The correct answer set “3-11-12-20-21-32-40” therefore shows 2 instances of consecutive correct answers. The number of consecutive correct answers score can be obtained, according to Equation (10), by dividing the count of consecutive answers by the number of correct answers. As is clear, the number of correct responses in “3-11-12-20-21-32-40” is 7. The number of consecutive correct answers score of 0.286 is in the rightmost column Table 3.

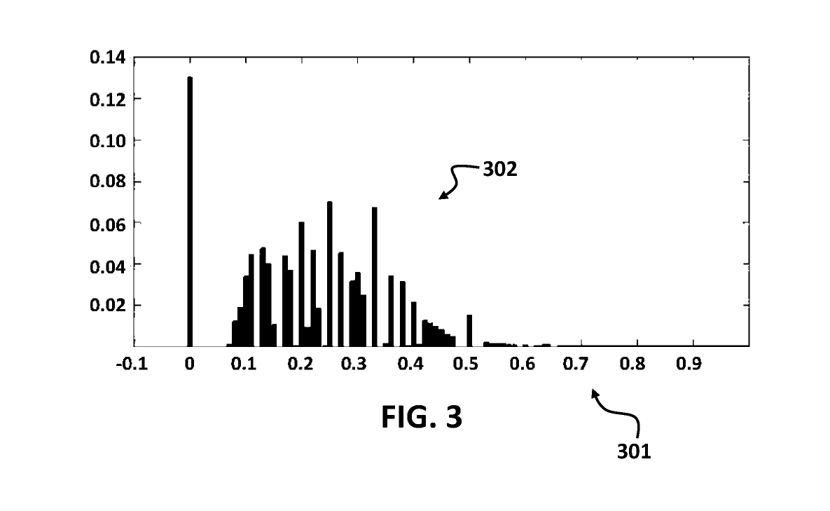

FIG. 3 shows probability of the occurrence of different values of the number of consecutive correct answers scores, each obtained by the Equation (10) calculations of discussed hereinabove, using an anosmic subject's responses a 40-item forced-choice odor identification test with 4-choice questions. Referring to FIG. 3, the horizontal axis 301 represents values of the number of consecutive correct answers scores, and the vertical black bars 302 represent their respective probabilities. As can be seen, for this example, it is highly probable that the number of consecutive correct answer score is between 0 and 0.4. Accordingly, for this example, the number of consecutive correct answers criteria can be set such that a consecutive correct answer score that is between 0 and 0.4 passes.

### Position of the First Correct Answer

As described above, one of the answer pattern criteria can be the position of the first correct answer score. In an aspect, this score can be utilized to exploit a correlation, identified by the present inventors, between answers being from a malingering subject and a later position of the first correct answer. The present inventors believe, without subscribing to any particular scientific theory, the correlation may be due to malingering subjects' fearing detection if their first correct answer is to an early-presented question.

If the answers are random, i.e., if the subject is anosmic, the probability of the first correct answer being to question k can be calculated using Equation (11) as follows:

$$P ( A ) = ( \frac{1}{Q} ) {( \frac{( Q - 1 )}{Q} )}^{k - 1} \qquad Equation ( 11 )$$

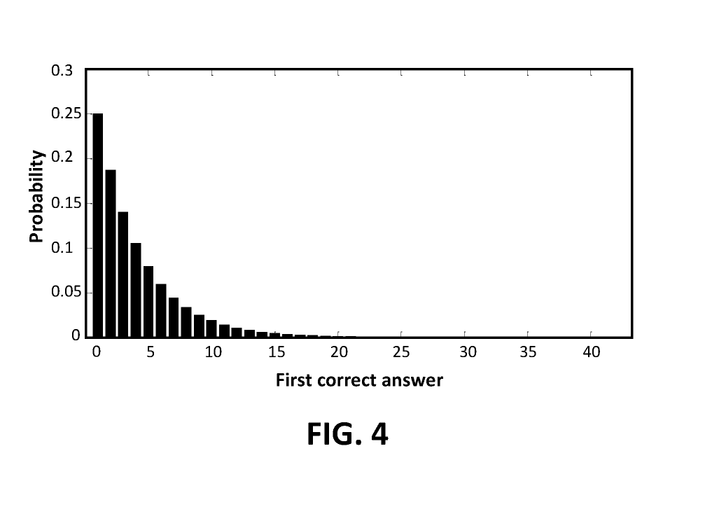

FIG. 4 shows the probability of the first correct answer being to question kin a 40-item forced-choice odor identification test, with 4-choice questions. The probabilities can be calculated using Equation (12) below, which is Equation (11), with Q being equal to 4:

$$P ( A ) = ( \frac{1}{4} ) {( \frac{( 3 )}{4} )}^{k - 1} \qquad Equation ( 12 )$$

As can be seen, the probability of the first question being answered correctly is 0.25. The probability of the first correct answer occurring after 10 questions—which is the probability of answering the first nine questions incorrectly—for this example, being 0.75 raised to the 9^{th} power. That value is approximately 0.025, which can be viewed as low.

Referring to FIG. 4, a range of 0 to 9 can be deemed the statistically likely value of the position of the first correct answer score, for an anosmic subject's responses to the example 40-item forced-choice odor identification test, with 4-choice questions. Therefore, for this example, the reference range for the position of the first correct answer score can be the range from 0 and 9. Therefore, the position of the first correct answer criterion can be the range from 0 to 9.

### Distribution of Correct Answers for a Specific Odorant

The distribution of correct answers score and related distribution of correct answers criterion described above are not specific to any particular odorant. In an aspect, a coefficient of variation can be calculated for the correct answers provided by the subject, when they are presented with a specific odorant in the test. The coefficient of variation is defined herein as the ratio of the standard deviation of the number of correct answers for a specific odorant to the average number of correct answers for each odorant.

For example, in a 40-item forced-choice odor identification test with 4-choice questions, where 5 odorants are presented to the subject in a manner such that each odorant is repeated 8 times throughout the test, the coefficient of variation can be defined as the ratio of the standard deviation of the number of correct answers for a specific odorant, to the average number of correct answers for each odorant in the test. The coefficient of variation can be calculated using equations similar in form to Equations (3)-(6) but, for convenience and clarity, are presented below as Equations (13)-(16):

$$\begin{matrix}Cv = \frac{σ}{\overline{x}} & Equation ( 13 ) \\ 0 \leq Cv \leq {OD}^{1 / 2} & Equation ( 14 ) \\ \overline{x} = \frac{1}{ND} {\sum }_{i = 1}^{ND} {x}_{i} & Equation ( 15 ) \\ σ = {( \frac{1}{ND} {\sum }_{i = 1}^{ND} {( {x}_{i} - \overline{xd} )}^{2} )}^{\frac{1}{2}} & Equation ( 16 )\end{matrix}$$

where Dᵥₒ represents the coefficient of variation, ND represents the number of odorants; x̅d̅ represents the average number of correct answers; and xd represents the number of correct answers for a specific odorant. In an aspect 5 odorants can be presented to the subject with a predefined repetition pattern. For purposes of description, the 5 odorants are referred to as Odorant 1, Odorant 2, Odorant 3, Odorant 4, and Odorant 5.

In a 40-item forced-choice odor identification test, each of the 5 odorants is repeated 8 times throughout the test. Therefore, the minimum number of correct answers for a specific odor is zero and the maximum number of correct answers for an odorant is 8.

The sample space, SDO, for all possible numbers of correct answers for each specific odorant, assuming a 40-item forced-choice odor identification test, each of 5 odorants repeated 8 times, can be calculated using the following Equation (17):

SDO={(y₁,y₂,y₃,y₄,y₅)|0≦yᵢ≦U,Σᵢ₌^{ND}yᵢ≠0} Equation (17)

where y₁ represents the number of correct answers for Odorant 1, y₂ represents the number of correct answers for Odorant 2, y₃ represents the number of correct answers for Odorant 3, y₄ represents the number of correct answers for Odorant 4, and y₅ represents the number of correct answers for Odorant 5.

The probability of each set to occur is calculated using Equations (18) and (19),

$$\begin{matrix}{set}_{jth} = \{ {( {y}_{1} , {y}_{2} , {y}_{3} , {y}_{4} , {y}_{5} )}_{i} \} i = 1 , 2 , 3 … N & Equation ( 18 ) \\ P ( {set}_{jth} ) = {\sum }_{i = 1}^{N} \{ {\prod }_{i = 1}^{ND} ( \frac{8}{{y}_{k}^{i}} ) {( \frac{1}{4} )}^{k} {( \frac{3}{4} )}^{8 - k} \} & Equation ( 19 )\end{matrix}$$

A reference range for the coefficient of variation can be obtained by calculating the probability of each coefficient of variation to happen in cases, where all questions are answered randomly. For example, for a 40-item forced-choice odor identification forced-choice odor identification test with 4-choice questions, where 5 odorants are presented to the subject and each odorant is repeated 8 times throughout the test, a coefficient of variation between 0 and 0.95 is highly probable based on probability calculations in an anosmic subject, who answers all questions randomly. Accordingly, the reference range for the distribution of correct answers for a specific odorant is between 0 and 0.95.

It will be understood that there is no limit in the number of odorants presented to the subject, although, preferably, each odorant or some odorants is repeated at least 2 times over the course of the test.

### Number of Similar Wrong Answers Chosen for a Specific Odorant

In an aspect, operations at 101 can be configured such that predesigned sets of wrong answers or distracters can be presented along with the correct answer for each odorant. For example, each odorant can be repeated at least 2 times, each time using the same distracters. This aspect can enable exploitation of a statistical tendency, identified by the present inventors, of low probability for test results from an anosmic subject to select the same specific wrong alternative or distracter whenever he or she smells a specific odorant. This aspect can also exploit a statistical likelihood, identified by the present inventors, of test results from malingering subjects showing a likely selection of the same specific wrong answer whenever they smell a specific odorant.

According to one implementation, for a 40-item odor identification test with 4-choice questions, in which 5 odorants are presented to the subject, where each odorant is repeated 8 times throughout the test, 2 types of questions, can be designed for each odorant. In each type, 3 specific distracters or wrong alternatives is presented to the subject. In this implementation, the maximum number of similar wrong answers that can be chosen for a specific odorant is 4. To score the number of similar wrong answers chosen for a specific odorant, the following example operations can be applied: assigning a score of 1 to instances of 3 similar wrong answers being chosen by the subject for a specific odorant; and assigning a score of 2 to instances of 4 similar wrong answers being chosen by the subject for a specific odorant. In an aspect, operations can further include generating the number of similar wrong answers chosen for a specific odorant score as the sum of these scores.

Referring to the example generation of the number of similar wrong answers chosen for a specific odorant score that is described above, one general example can assigning a score of A to instances of D similar wrong answers being chosen by the subject for a specific odorant, and assigning a score of A+1 to instances of D+1 similar wrong answers being chosen by the subject for a specific odorant, generating, based at least in part on a sum of A and A+1, a number of similar wrong answers chosen for a specific odorant score. In the above-described example, the value of “A” is 1 and the value of “D” is 3.

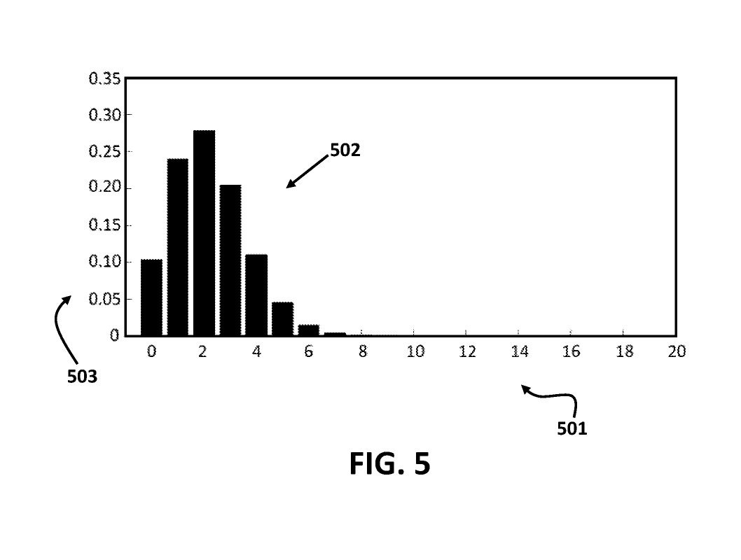

FIG. 5 shows one example probability density function of the number of similar wrong answers score for a specific odorant, by an anosmic subject. The scores are shown on the horizontal axis 501, probabilities of the total scores are shown by black bars 502, and their corresponding numerical values are shown on the vertical axis 503. As can be seen in FIG. 5, for this example, it can be likely for an anosmic subject's test results to show a range of 0 to 4 in the number of similar answers chosen for a specific odorant score. The range of 0 to 4 can therefore be an example “reference range” for the number of similar wrong answers chosen for a specific odorant criterion—assuming the 40-item odor identification test with 4-choice questions where 5 odorants are presented to the subject and each odorant is repeated 8 times throughout the test.

It will be understood that upon completion of operations at 102, a process according to one aspect may have obtained a number of correct answers score and an answer pattern score. As described above, the answer pattern score can, in an aspect, include a “failed criteria count.” For example, if answer pattern criteria are configured to include the distribution of correct answers criterion and the number of consecutive correct answers criteria, a subject's answers failing both of these criteria can result in a “failed criteria count” of 2. As another example, if the answer pattern criteria are configured to include the distribution of correct answers criterion, the number of consecutive correct answers criterion, and the position of the first correct answer criterion, a subject's answers failing any one of these criteria will result in a failed criteria count of 1. The subject's answers failing any 2 of these criteria and passing the remaining criterion (among these 3) will result in a failed criteria count of 2.

Exemplary Operations in Classifying the Subject

In an aspect, upon completion of the above-described operations at 102, operation can be applied that classify the subject according to olfactory condition type. The olfactory condition type can being a member of an olfactory condition set. In an aspect, the olfactory condition set can include anosmic and malingering. In another aspect, the olfactory condition set can include anosmic, normal, and malingering. In another aspect, the olfactory condition set can include anosmic, normal, malingering and micro.

In an aspect, operations at 103 in classifying the subject according to olfactory condition type can be based, at least in part, on a combination of the number of correct answers score and the answer pattern score.

### EXAMPLES

The following examples represent methods and techniques for carrying out aspects of the present application. It should be understood that numerous modifications can be made without departing from the intended scope of the disclosure.

### Example 1

### Conducting One Forced-Response Odor Identification Test

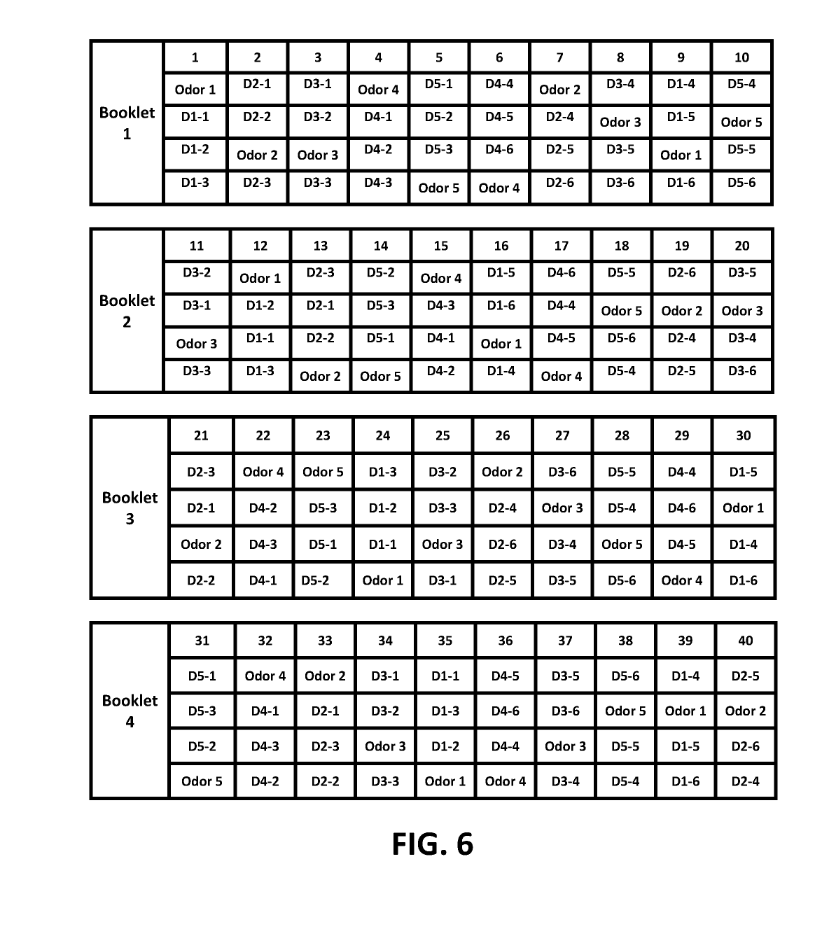

FIG. 6 shows an example of alternatives for each question. In the figure, the correct answer to each question is identified by the word “odor,” followed by a number that designates one of the 5 odorants. For example, referring to FIG. 6, the correct answer to question 1 is odor 1, and the correct answer to question 8, is odor 3. In an aspect, for each odorant there may be two types of questions. In each of the two types, the list of alternative choices includes the correct answer, and 3 alternative choices. The 3 alternative choices are wrong alternatives or distracters. The two types present respectively different wrong alternatives. The wrong alternatives for each odorant are designated in FIG. 6 by letter D followed by the corresponding odorant number.

Referring to FIG. 6, all wrong alternatives for odorant 1 are designated as D1; all wrong alternatives for odorant 2 are designated as D2; all wrong alternatives for odorant 3 are designated as D3; all wrong alternatives for odorant 4 are designated as D4; and all wrong alternatives for odorant 5 are designated as D5. Six wrong alternatives are presented for each odorant, which are coded with numbers, 1 to 6. For example, for odorant 1, 6 wrong alternatives of D1-1, D1-2, D1-3, D1-4, D1-5, and D1-6 are presented in the test. In an aspect, different wrong alternatives can be used for each odorant.

In an aspect, the questions in the other 3 booklets can have the same wrong alternatives presented for each odorant. However, in a further aspect, the arrangement of the correct answer and wrong alternatives presented in each item can be different in each booklet.

### Example 2

### Scoring the Odor Identification Test

Upon receiving all of the subject's answers to all the questions in the odor identification test, for example, as described above, operations such as the FIG. 1 operations 102 can score the subject's answers. In an aspect, the test results are scored based on a set of criteria. In this criteria include a number of correct answers criterion, distribution of correct answers criterion, number of consecutive correct answers criterion, position of the first correct answer criterion, distribution of correct answers for a specific odorant criterion, and number of similar wrong answers chosen for a specific odorant criterion.

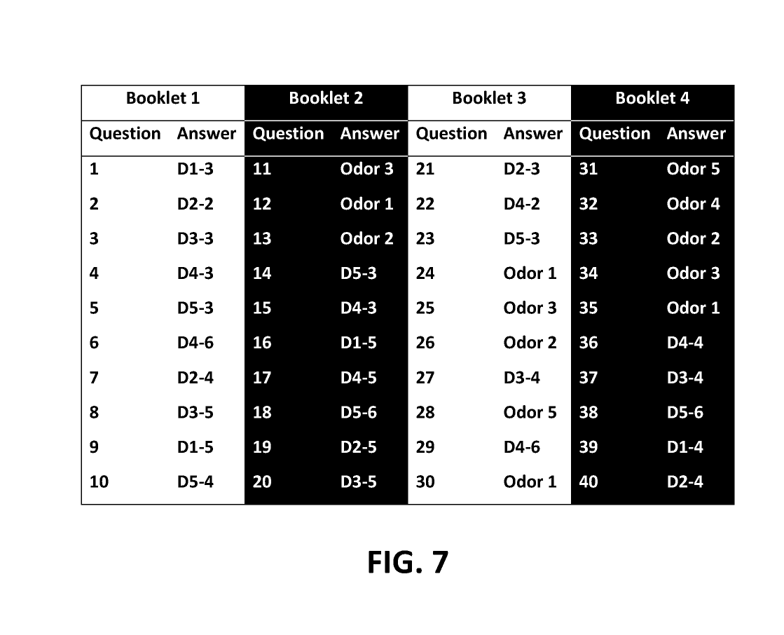

FIG. 7 illustrates the answer sheet of an exemplar subject. In this figure, the question numbers are presented in columns labeled as “Question” and the answers provided by the subject are presented in columns labeled as “Answer”. As described above, FIG. 6 illustrates how the alternatives are designed for each question. Based on this design, the answer sheet of the subject can be scored. Referring to FIG. 7, correct answers are designated by the odorant's name: Odor 1, Odor 2, Odor 3, Odor 4, or Odor 5; and wrong answers are designated by the name of the distracter, which is chosen by the subject. For example, in question 6, the odorant presented to the subject is Odor 4, but the subject has chosen distracter D4-6, and for example, in question 28, Odor 5 is presented to the subject, and the subject has chosen the correct answer, which is Odor 5.

Referring to the answer sheet illustrated in FIG. 7, the subject has answered 13 questions correctly, therefore the number of correct answers score is 13. Regarding the distribution of correct answers in each booklet, i.e., to each group of 10 questions, the subject has zero correct answers in the first booklet, 3 correct answers in the second booklet, 5 correct answers in the third booklet, and 5 correct answers in the fourth booklet. The coefficient of variation can be calculated as the variance of number of correct answers in each booklet ({0, 3, 5, 5}) divided by the average of {0, 3, 5, 5}. The coefficient of variation in this case is equal to 0.73, and therefore the distribution of correct answers score is 0.73.

Regarding the number of consecutive correct answers, operations at 102 detect that the subject's answers to questions 11, 12, 13, 24, 25, 26, 28, 30, 31, 32, 33, 34, and 35 are correct. The operations at 102 detect, in these answers, 9 pairs of consecutive correct answers. As described above in reference to Equation (10), operations at 102 can calculate the number of consecutive correct answers score by dividing the number of consecutive correct answers by the total number of correct answers (9/13). Therefore, for this example, the number of consecutive correct answers score equals 0.69.

Regarding the position of the first correct answer score, operations at 102 can detect the first correct answer being in response question number 11 and, therefore, can determine the position of the first correct answer score to be 11.

Regarding the distribution of correct answers for a specific odorant, the subject's responses show 4 correct answers for odorant 1, 3 correct answers for odorant 2, 3 correct answers for odorant 3, 1 correct answer for odorant 4, and 2 correct answers for odorant 5. Operations at 102 can therefore calculate the coefficient of variation can therefore be calculated as the standard deviation of {4, 3, 3, 1, 2} divided by the average of {4, 3, 3, 1, 2}. Therefore, the score of distribution of correct answers for a specific odorant is 0.44.

Regarding the number of similar wrong answers chosen for a specific odorant, the subject has chosen the distracter D5-3 three times, which leads to a score of 1 for the criterion of number of similar answers chosen for a specific odorant.

### Example 3

### Classifying the Subject Based on the Scores

Table 4 shows example scores of the subject's answers, as discussed in reference to connection with Example 2, along with the reference ranges defined for each criterion. As can be seen in this table, the subject has performed within the reference ranges for criteria of number of correct answers, distribution of correct answers, distribution of correct answers for a specific odorant, and number of similar wrong answers chosen for a specific odorant. The subject, according to results presented in this table, has failed the criteria of number of consecutive correct answers and the position of the first correct answer.

If the classification were based solely on the number of correct answers, such the known UPSIT, the subject would be diagnosed as anosmic. However, operations according to this disclosure also score the subject's responses according to at least one answer pattern criterion. Example answer pattern criteria, as described above, can include at least one from among distribution of correct answers criterion, number of consecutive correct answers criterion, position of the first correct answer criterion, distribution of correct answers for a specific odorant criterion, and number of similar wrong answers chosen for a specific odorant criterion. As described in greater detail in reference to FIG. 8, operations according to various aspects can further classify the subject using, in combination with the number of correct answer, the at least one answer pattern criterion. As will also be described, the further classification can detect a malingering subject, who may have been misidentified by conventional techniques, such as UPSIT

TABLE 4
Scores obtained by the subject and reference ranges used for each criterion

| | | Reference |
|---|---|---|
| Criterion | Score | Range |
| Number of correct answers | 13 | [6 14] |
| Distribution of correct answers | 0.73 | [0 0.86] |
| Number of consecutive correct answers | 0.69 | [0 0.4] |
| Position of the first correct answer | 11 | [0 9] |
| Distribution of correct answers for a specific odorant | 0.44 | [0 0.95] |
| Number of similar wrong answers chosen for a specific | 1 | [0 4] |
| odorant | | |

FIG. 8 illustrates one exemplary decision scheme for classifying the subject based on scores of the odor identification test, such as described in relation to Example 1. Referring to FIG. 8, one example decision scheme as be as follows:
i) if a subject's answer have less than 6 that are correct, the subject is classified as malingering, regardless of the failed criteria count;
ii) if the subject has 6 correct answers, and the failed criteria count is
   more than 2, the subject can be classified as “highly suspected” of malingering, and
   less than or equal 2, a notice may generated suggesting or indicating that a retest is required;
iii) if the subject has 7 correct answers, and the failed criteria count is
   a zero, the subject can be classified as “highly suspected” of being anosmic,
   1 or 2, the notice may generated suggesting or indicating that retest is required, and then a retest is required, and
   greater than 2, the subject may be classified as highly suspected of malingering;
iv) if the subject has between 7 and 14 correct answers, and the failed criteria count is
   zero, the subject can be classified as highly suspected of being anosmic,
   1 or 2, the notice may generated suggesting or indicating retest is required, than
   greater than 2 the subject can be classified as highly suspected of malingering;
v) if the subject has between 15 and 34 correct answers, the subject can be classified as suspected of having microsmia; and
vi) if the subject has more than 34 correct answers, the subject can classified as normosmic

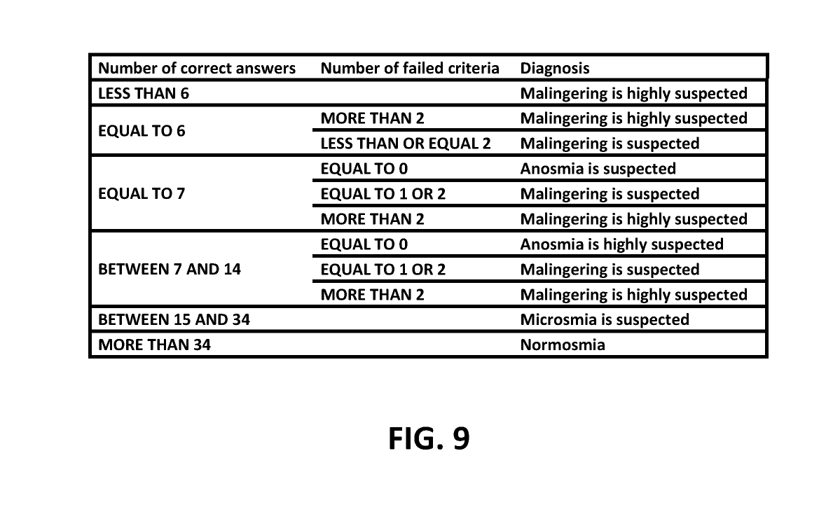

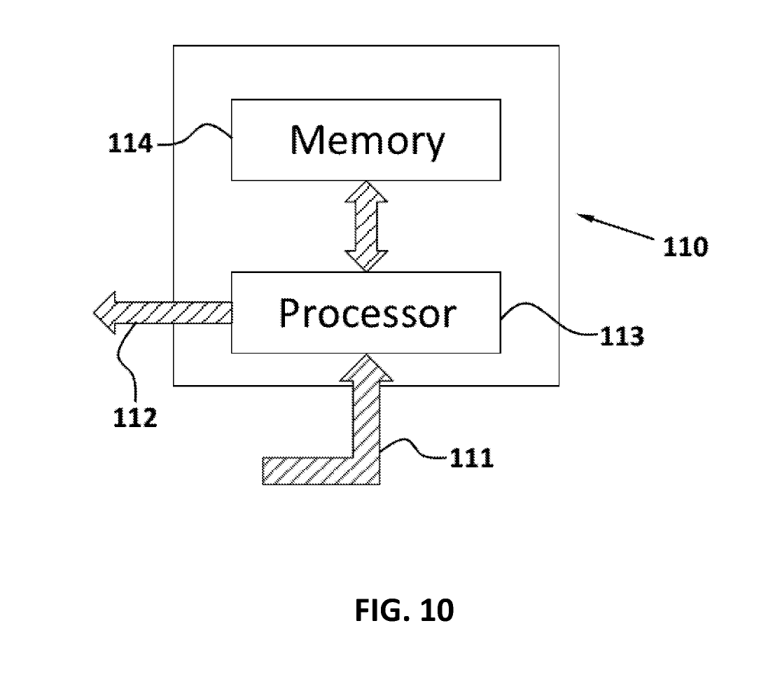

FIG. 9 illustrates another exemplary decision scheme for classifying the subject based on scores of the odor identification test, such as described in relation to Example 1. Referring to FIG. 9, the decision scheme as be as follows:
vii) if a subject's answers have less than 6 that are correct, the subject is classified as malingering, regardless of the failed criteria count;
viii) if the subject has 6 correct answers, and
   the failed criteria count is more than 2, the subject can be classified as “highly suspected” of malingering, and
   if the failed criteria count is less than or equal 2, the subject can be classified as “suspected” of malingering a notice;
ix) if the subject has 7 correct answers, and the failure count is
   zero, the subject can be classified as anosmic, or as “highly suspected” of being anosmic,
   1 or 2, the subject may be classified as suspected of malingering, and
   greater than 2, the subject may be classified as highly suspected of malingering;
x) if the subject has between 7 and 14 correct answers, and the failed criteria count is
   zero, the subject can be classified as highly suspected of being anosmic,
   1 or 2, the subject can be classified as suspected malingering,
   greater than 2 the subject can be classified as highly suspected of malingering;
xi) if the subject has between 15 and 34 correct answers, the subject can be classified suspected of having microsmia; and
xii) if the subject has more than 34 correct answers, the subject can classified as normosmic.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

Except as stated immediately above, nothing that has been stated or illustrated is intended or should be interpreted to cause a dedication of any component, step, feature, object, benefit, advantage, or equivalent to the public, regardless of whether it is or is not recited in the claims

The Abstract of the Disclosure is provided to allow the reader to quickly ascertain the nature of the technical disclosure. It is provided with the understanding that it will not be used to interpret or limit the scope or meaning of the claims. In addition, in the foregoing Detailed Description, it can be seen that various features are grouped together in various implementations for the purpose of streamlining the disclosure. This is not to be interpreted as reflecting an intention that the claimed implementations require more features than are expressly recited in each claim. Rather, as the following claims reflect, inventive subject matter lies in less than all features of a single disclosed implementation. Thus the following claims are hereby incorporated into the Detailed Description, with each claim standing on its own as a separately claimed subject matter

What is claimed is:

1. A method for detecting olfactory malingering, the method comprising:
presenting a subject a forced-choice odor identification test;
receiving the subject's answers;
scoring the subject's answers, based at least in part on identifying each of the subject's answers as correct or incorrect, according to a number of correct answers score and an answer pattern score; and
classifying the subject according to an olfactory condition type, the olfactory condition type being a member of an olfactory condition set, the olfactory condition set including a malingering type, the classifying being based at least in part on a combination of the number of correct answers score and the answer pattern score.

2. The method of claim 1, wherein classifying the subject according to olfactory condition type includes classifying the subject, based at least in part on the answer pattern score, as being suspected of belonging to one olfactory condition type among the set of olfactory condition types, and being highly suspected of belonging to the one olfactory condition type.

3. The method of claim 1, wherein generating the answer pattern score comprises:
calculating a set of answer pattern values, the set of answer pattern values indicating respective levels of existence, in the subject's answers, of each among a corresponding set of answer patterns; and
comparing the set of answer pattern values to a corresponding set of answer pattern criteria, and generating, in response, a failed criteria count, wherein the answer pattern score includes the failed criteria count.

4. The method of claim 3, wherein classifying the subject according to an olfactory condition type comprises:
upon determining that the number of correct answers score is within a number of correct answers first reference range, classifying the subject as the malingering type, wherein the method further comprises:
upon determining a conjunction of the number of correct answers score being within a number of correct answers second reference range, and the failed criteria count exceeding a threshold, generating a retest notice.

5. The method of claim 4, wherein classifying the subject according to olfactory condition type further comprises:
upon determining a conjunction of the number of correct answers score being within the number of correct answers second reference range, and the failed criteria count not exceeding the threshold, classifying the subject as the malingering type.

6. The method of claim 5, wherein the threshold is a first threshold, and wherein olfactory condition set further includes an anosmia type, and wherein classifying the subject according to an olfactory condition type further comprises:
upon determining that a conjunction of the number of correct answers score being within a number of correct answers third reference range and the failed criteria count being in a given minimum range, classifying the subject as likely to be the anosmia type; and
upon determining that a conjunction of the number of correct answers score being within the number of correct answers third reference range and the failed criteria count being above a second threshold, in a given minimum range, classifying the subject as the malingering type.

7. The method of claim 6, wherein the number of correct answers second reference range exceeds the number of correct answers first reference range, and the number of correct answers third reference range exceeds the number of correct answers second reference range

8. The method of claim 6, wherein the given minimum range is zero.

9. The method of claim 3 wherein presenting the subject the odor identification test, receiving the subject's answers, and identifying each of the subject's answers as correct or incorrect comprises presenting the subject a plurality of groups of odorant sample/response questions, and wherein the calculating the answer pattern score comprises:
counting the total number of correct answers in the subject's answers to the odorant sample/response questions,
determining a coefficient of variation, wherein the coefficient of variation indicates a variation in correct answers in the subjects' answers to the different groups of odorant sample/response questions, and
comparing the coefficient of variation to a given distribution of correct answers criterion, and
upon the coefficient of variation failing the distribution of correct answers criterion, adding one criteria fail to the answer pattern score.

10. The method of claim 9, wherein the set of pattern criteria includes a position of first correct answer score, wherein generating the answer pattern score comprises:
comparing the position of first correct answer score to a position of first correct answer criterion and, upon the position of first correct answer score failing to meet the position of first correct answer criterion, incrementing the failed criteria count by one.

11. The method of claim 10 wherein the set of pattern criteria includes a number of consecutive correct answers score, wherein generating the answer pattern score comprises:
comparing the number of consecutive correct answers score to a number of consecutive correct answers criterion and, upon the number of consecutive correct answers score failing to meet the number of consecutive correct answers criterion, incrementing the failed criteria count by one.

12. The method of claim 3, wherein presenting the subject the forced-choice odor identification test is configured to present to the subject a set of odorants, in a manner such that each odorant in the set of odorants is presented at least twice, and
wherein scoring the subject's answers includes calculating a coefficient of variation, wherein the coefficient of variation is defined as the ratio of a standard deviation of the number of correct answers for a specific odorant, to the average number of correct answers for each odorant in the set of odorants.

13. The method of claim 3, wherein presenting the subject the forced-choice odor identification test is configured to present to the subject a set of odorants, in a manner such that each odorant in the set of odorants is presented at least twice, and wherein in each of the at least two presentations, the subject is presented the same list of alternative choices for the subject to select from, and wherein the list of the alternative choices includes a name of the odorant presented, and the same names for other alternative choices in the list.

14. The method of claim 13, wherein scoring the subject's answers comprises:
assigning a score of A to instances of D similar wrong answers being chosen by the subject for a specific odorant, and assigning a score of A+1 to instances of D+1 similar wrong answers being chosen by the subject for a specific odorant; and
generating, based at least in part on a sum of A and A+1, a number of similar wrong answers chosen for a specific odorant score.

15. A method for detecting olfactory malingering, the method comprising:
presenting a subject a forced-choice odor identification test, receiving the subject's answers, and identifying each of the subject's answers as correct or incorrect;
generating, based at least in part on identifying each of the subject's answers as correct or incorrect, a number of correct answers score and at least one from among a number of consecutive correct answers score and a position of the first correct answer score; and
classifying the subject according to olfactory condition type, the olfactory condition type being a member of an olfactory condition set, the olfactory condition set including a malingering type, the classifying being based at least in part on a combination of the number of correct answers score and a comparison of the number of consecutive correct answers score to a number of consecutive correct answers criterion, or a comparison of the position of the first correct answer score to a position of the first correct answer score criterion, or both.

16. The method of claim 15, wherein presenting the subject the odor identification test, receiving the subject's answers, and identifying each of the subject's answers as correct or incorrect comprises presenting the subject a plurality of groups of questions, and wherein the method further comprises:
counting the total number of correct answers in the subject's answers to the questions; and
determining a coefficient of variation, wherein the coefficient of variation indicates a variation in correct answers in the subjects' answers to the different groups of questions, and
wherein the classifying is further based at least in part on comparing the coefficient of variation to a given distribution of correct answers criterion.

17. The method of claim 16, wherein the method further comprises generating a position of first correct answer score, wherein the classifying is further based at least in part on comparing the position of first correct answer score to a given position of first correct answer criterion.

18. The method of claim 17, wherein the method further comprises generating a number of consecutive correct answers score, and wherein the classifying is further based at least in part on comparing the number of consecutive correct answers score to a given number of consecutive correct answers criterion.

19. A data processing system for detecting olfactory malingering, the system comprising:
a processor; and
a memory storing executable instructions for causing the processor to:
receive a subject's answers to an odor identification test; and
score the subject's answers, based at least in part on identifying each of the subject's answers as correct or incorrect, according to a number of correct answers score and an answer pattern score; and
classify the subject according to an olfactory condition type, the olfactory condition type being a member of an olfactory condition set, based at least in part on a combination of the number of correct answers score and the answer pattern score, wherein the olfactory condition set includes a malingering type.

20. The method of claim 19, wherein the memory further stores executable instructions for causing the processor to:
calculate a set of answer pattern values, the set of answer pattern values indicating respective levels of existence, in the subject's answers, of each among a corresponding set of answer patterns; and
compare the set of answer pattern values to a corresponding set of answer pattern criteria, and generating, in response, a failed criteria count, wherein the answer pattern score includes the failed criteria count.
